(19) United States
(12) Patent Application Publication
(10) Pub. No.: US 2025/0345542 A1
(43) Pub. Date: Nov. 13, 2025

(54) AUTOMATED ENDOTRACHEAL INTUBATION DEVICE

(71) Applicants: Hamdy ELSAYED-AWAD, Columbus, OH (US); Mark a. RUEGSEGGER, Columbus, OH (US); Ohio State Innovation Foundation, Columbus, OH (US)

(72) Inventors: Mark A. RUEGSEGGER, Dublin, OH (US); Hamdy ELSAYED-AWAD, Columbus, OH (US); Michael NAPOLI, Columbus, OH (US); Taggart STORK, Bellbrook, OH (US); Connor GANTT, Columbus, OH (US); Hossam MONTASSER, Dublin, OH (US); Nathan AMES, Sunbury, OH (US); Ethan HOPPING, Columbus, OH (US); Tim MILES, Grove City, OH (US); Christian LOPEZ VOGLER, Columbus, OH (US); Jordan PEIFFER, Camp Hill, PA (US)

(21) Appl. No.: 18/860,140

(22) PCT Filed: Apr. 26, 2023
(86) PCT No.: PCT/US2023/020028

§ 371 Date: Oct. 25, 2024

Related U.S. Application Data
(60) Provisional application No. 63/334,908, filed on Apr. 26, 2022.
(60) Provisional application No. 63/454,828, filed on Mar. 27, 2023.

(51) Int. Cl. A61M 16/04; A61B 1/04

(52) U.S. Cl. CPC A61M 16/0402; A61B 1/04; A61M 2205/02; A61M 2205/36; A61M 2205/502; A61M 2210/1032

(57) ABSTRACT

Various implementations include an automated endotracheal intubation device, including: a flexible tube sized to be advanced within a patient's airway; a base system; and a deployment arm having a proximal end coupled to the base system and a distal end spaced apart from the proximal end, the deployment arm including: at least one arm segment coupled to and extending from the base system, the at least one arm segment defining a channel within which the flexible tube is disposed; and an end effector coupled to the distal end of the deployment arm, the end effector including one or more twisted and coiled polymer (TCP) tendons configured to controllably expand and contract upon application of heat.

### CROSS-REFERENCE TO RELATED APPLICATIONS

This application claims benefit of U.S. Provisional Application No. 63/334,908, filed Apr. 26, 2022, and U.S. Provisional Application No. 63/454,828, filed Mar. 27, 2023, which are hereby incorporated herein by reference in their entireties.

### BACKGROUND

Endotracheal intubation is a commonly performed procedure that is critical to the health and safety of patients. The procedure's purpose is to give access to and maintain the patient's airway so that their breathing can be safely regulated. This is done in emergency situations where a patient's breathing may be unstable and planned procedures where general anesthesia is used. An endotracheal intubation tube, as shown in FIG. 1A, is used to route air from outside the body, likely from a mechanical ventilator to the trachea and thus the lungs. The process of endotracheal intubation refers to the placement of the tracheal tube. There are three common methods used clinically to place the tracheal tube: direct laryngoscopy, video laryngoscopy, and flexible intubation scope, each shown in FIG. 1B. The placement of the tracheal tube needs to be quick as the gas exchange in the lungs is vital to survival, yet it has a risk of causing major damage to the patient, such as oxygen desaturation, laryngospasm, pneumothorax, and severe cases can even cause brain death. The current issues with this process revolve around the varying amount of training needed for conducting endotracheal intubation, as it is performed in a variety of circumstances by multiple personnel, the selection of outdated and harder to use tools to assist in the placement of the tracheal tube, and anatomical differences between people that makes for difficult visualization of anatomical features and thus increases the difficulty in properly placing the tracheal tube. There is a clinical need to develop new devices, systems, and methods of placing the tracheal tube in patients that is easier to perform, is more reliable, and increases the safety of the patient.

### SUMMARY

Various implementations include an automated endotracheal intubation device, including: a flexible tube sized to be advanced within a patient's airway; a base system; and a deployment arm having a proximal end coupled to the base system and a distal end spaced apart from the proximal end, the deployment arm including: at least one arm segment coupled to and extending from the base system, the at least one arm segment defining a channel within which the flexible tube is disposed; and an end effector coupled to the distal end of the deployment arm, the end effector including one or more twisted and coiled polymer (TCP) tendons configured to controllably expand and contract upon application of heat.

In some implementations, a device, wherein the base system includes: a stabilizer including at least one of a handheld brace, a c-clamp brace, and a stabilization board dimensioned to accommodate a patient's head.

In some implementations, a device, wherein the base system includes: a motor in electrical communication with a power source; and a driver coupled to the motor and the deployment arm, the driver configured to extend and retract the deployment arm with respect to the base system.

In some implementations, a device, wherein the base system further includes a control system including a user interface, the control system configured to receive input from a user on the user interface and control the deployment arm and the driver in response to signals sent from the control system to the device.

In some implementations, a device, wherein the driver includes a belt-driven system or a wheel-driven system.

In some implementations, a device, further including a camera coupled to the end effector; and a screen in electrical or wireless communication with the camera, wherein the control system is configured to display on the screen the location of the deployment arm and/or end effector within a patient's airway.

In some implementations, a device, wherein the at least one arm segment includes a plurality of arm segments, each of the plurality of arm segments including one or more twisted and coiled polymer (TCP) tendons.

In some implementations, a device, wherein the deployment arm includes one or more twisted and coiled polymer (TCP) tendons extending along the at least one arm segment or the end effector, the one or more TCP tendons configured to controllably move the at least one arm segment or the end effector between a neutral configuration and a curved configuration with 1 degree of freedom.

In some implementations, a device, wherein the deployment arm includes a plurality of twisted and coiled polymer (TCP) tendons extending along the at least one arm segment or the end effector, the plurality of TCP tendons configured to controllably move the at least one arm segment or the end effector between the neutral configuration and a plurality of curved configurations with at least 2 degrees of freedom.

In some implementations, a device and 9, wherein the driver configured to extend and retract the deployment arm with respect to the base system and the plurality of TCP tendons operate simultaneously to move the deployment arm or the end effector in at least 3 degrees of freedom to navigate the patient's airway.

Various other implementations include a method of automated endotracheal intubation, the method including: providing an intubation device including a base system and a deployment arm coupled to the base system, the base system including a driver system for extension, and the deployment arm including a twisted and coiled polymer (TCP) end effector; inserting the deployment arm into a patient's mouth adjacent to an airway; activating the driver system to extend the deployment arm further into the airway; activating the twisted and coiled polymer (TCP) end effector to navigate the patient's anatomy; deploying a flexible tube configured to maintain the patient's airway; retracting the end effector and the deployment arm out of the patient's airway.

In some implementations, a method, further including placing the stabilizer underneath a patient's head.

### BRIEF DESCRIPTION OF DRAWINGS

Example features and implementations of the present disclosure are disclosed in the accompanying drawings. However, the present disclosure is not limited to the precise arrangements and instrumentalities shown. Similar elements in different implementations are designated using the same reference numerals.

FIG. 1A shows an example of an existing endotracheal intubation tube, according to one implementation.

FIG. 1B shows three common methods used clinically to place the tracheal tube: direct laryngoscopy, video laryngoscopy, and flexible intubation scope, according to various implementations.

FIG. 2A provides an image of an example automated endotracheal intubation device, according to one implementation.

FIG. 2B shows the end effector of the device of FIG. 2A in more detail, according to one implementation.

FIG. 2C shows a top and side view diagram of the end effector of FIGS. 2A and 2B.

FIG. 3 shows another example end effector having three TCP tendons, according to one implementation.

FIGS. 4A and 4B show different views of another example end effector having two TCP tendons, according to one implementation.

FIG. 5A shows a deployment arm and associated TCP tendons in a neutral configuration, according to one implementation.

FIG. 5B show the deployment arm of FIG. 5A in a curved configuration, according to one implementation.

FIG. 6A shows another example deployment arm having multiple arm segments with an end effector as the distal arm segment.

FIG. 6B shows a single arm segment of the deployment arm of FIG. 6A, according to one implementation. rigid end 518 and a second rigid end 520.

FIG. 7 provides a diagram of the base system and associated control system for the device, according to one implementation.

FIG. 8A shows a prototype device including a handheld stabilizer and a wheel-driven deployment mechanism, according to one implementation.

FIG. 8B shows a prototype device including a c-clamp brace and a belt-driven deployment mechanism, according to one implementation.

FIG. 8C shows a tripod stabilizer and a lead screw deployment mechanism, according to one implementation.

FIG. 8D shows an example of a rail driven deployment system for the base system, according to one implementation.

FIG. 9 shows a perspective and a side view of a hexagonal spine housing, according to one implementation.

FIGS. 10A and 10B show another prototypical implementation of the base system, according to one implementation.

FIG. 11 shows an implementation of the intubation device having a face mask portion engageable over a patient's mouth and/or nose, according to one implementation.

### DETAILED DESCRIPTION

Disclosed herein is an automated device, system, and method for endotracheal intubation including a base system and a deployment arm which takes advantage of twisted and coiled polymer actuators (TCA) or twisted and coiled polymers (TCP). Twisted and coiled polymers (TCP) are formed from a polymer fiber coiled into a helix. They act as an artificial muscle, expanding or twisting when heated (e.g., when heated above a glass transition temperature). The expansion produces an axial force and or a torsional force based only on the application heat (e.g., heating electrically, photonically, thermally, chemically, by absorption, or by other means). The TCP relaxes to the initial state when cooled and can be reheated to expand repeatedly.

In various embodiments, TCP artificial muscles can be produced through a twist insertion process. For example, a fiber (e.g., nylon or other polymer) can be twisted to the point of coiling. In another example, a fiber can be twisted nearly to the point of coiling and then wrapped around a mandrel or fiber or yarn core. Coiled thermal fiber or yarn actuators, in accordance with various embodiments, can be made via coiling from twisting to the point of writhe or snarling (self-coiled or coiled-by-twisting), via coiling around a mandrel or other suitable material that serves as a core about which the fiber or fibers can be wound (coiled-by-wrapping), or other suitable method. In various examples, such a core can be removable in part or in whole, including removal via dissolving.

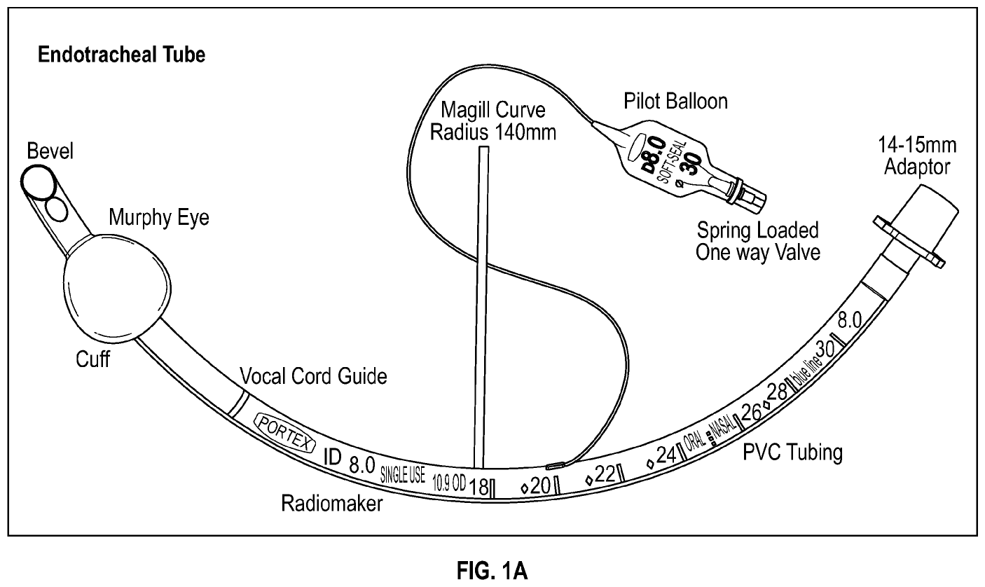

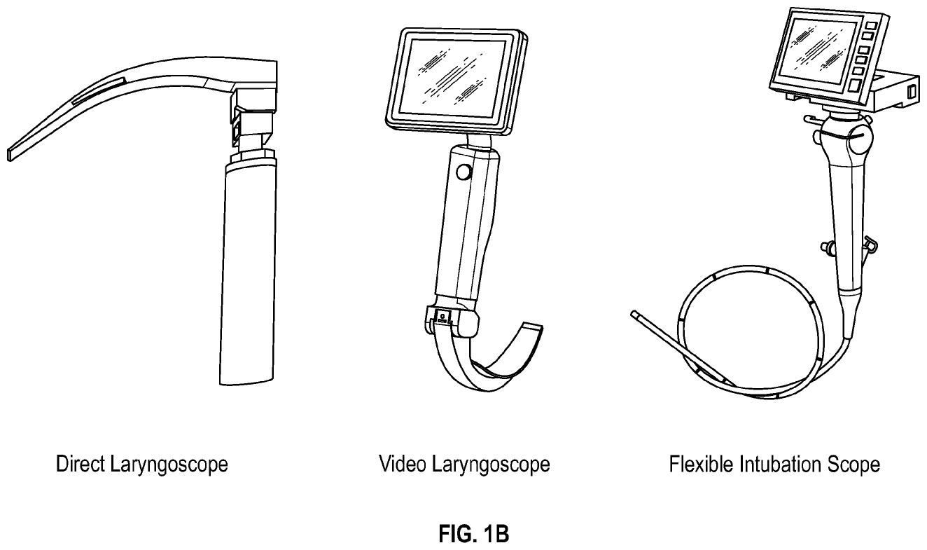

FIG. 1A shows an example of an existing endotracheal intubation tube, according to one implementation. FIG. 1B shows three common methods used clinically to place the tracheal tube: direct laryngoscopy, video laryngoscopy, and flexible intubation scope, according to various implementations. Rather than traditional, manual intubation, the disclosed device may be placed over a patient's face/mouth to automatically extend and navigate through the airway. The device may be controlled by a medical professional as it navigates the airway, or it may be trained using artificial intelligence and machine learning such that it recognizes the correct location to complete the intubation.

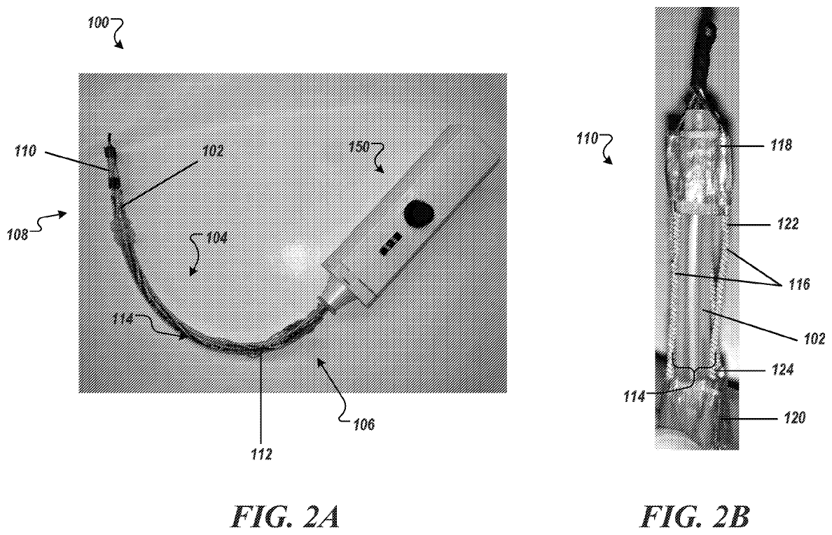

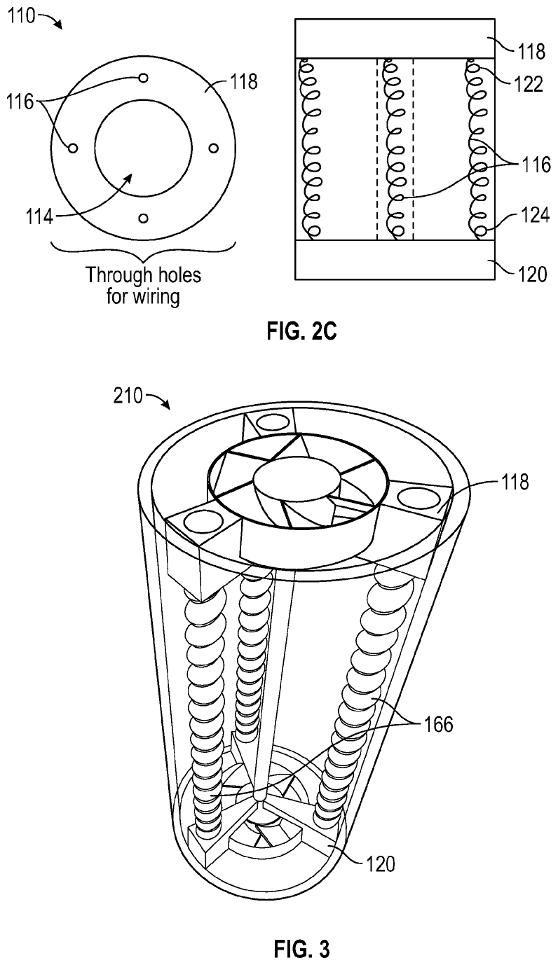

Generally, the twisted and coiled polymer (TCP) tendons in the end effectors and/or arm segments of the various deployment arms of FIGS. 2A-6B provide for controlled movement as the deployment arm navigates a patient's airway. For example, the end effector and adjacent arm segments may move between neutral and curved configurations to place the flexible tube within the patient's airway. In other words, the end effector can be curved to move in a radial direction with respect to the deployment arm.

The deployment arms and end effectors of FIGS. 2A-6B provide different variations on the structure and orientation of the TCP tendons, each of which results in variations on the motion of the end effector and/or deployment arm. For example, in some implementations, the deployment arm includes one or more TCP tendons extending along at least one arm segment or the end effector, wherein the one or more TCP tendons are configured to move the arm segment or the end effector with 1 degree of freedom. In other words, with one or two TCP tendons, a deployment arm and/or end effector may move radially back and forth in a single direction (e.g., an x-direction). In other implementations, the deployment arm includes a plurality of TCP tendons (e.g., 3 or 4 TCP tendons) extending along at least one arm segment or the end effector, wherein the plurality of TCP tendons is configured to move the arm segment or the end effector with 2 degrees of freedom. In other words, with three or more TCP tendons, a deployment arm and/or end effector may move radially in at least two directions (e.g., an x-direction and a y-direction).

FIG. 2A provides an image of an example automated endotracheal intubation device 100. The intubation device 100 includes a flexible tube 102 sized to be advanced within a patient's airway. Intubation device 100 further includes a base system 150 and a deployment arm 104.

The deployment arm 104 has a proximal end 106 and a distal end 108 spaced apart longitudinally from the proximal end 106. The proximal end 106 of the deployment arm 104 is coupled to the base system 150, and the distal end 108 of the deployment arm 104 is coupled to an end effector 110. The deployment arm 104 further includes at least one arm segment 112 coupled to and extending from the base system 150. The at least one arm segment 112 defines a channel 114 within which the flexible tube 102 is disposed. The end effector 110 includes one or more twisted and coiled polymer (TCP) tendons 116, the TCP tendons 116 configured to controllably expand and contract upon application of heat. Each TCP tendon 116 is in electrical communication with the base system 150 (e.g., via a wire extending along the deployment arm 104 from the proximal end 106 to the distal end 108).

The intubation device 100 of FIG. 2A includes one at least one arm segment 112 and one end effector 110, shown in more detail in FIG. 2B. Additionally, FIG. 2C shows a top and side view diagram of the end effector 110 of FIGS. 2A and 2B. The end effector 110 includes four TCP tendons 116, each disposed around the channel 114 equally spaced from each other (e.g., about 90 degrees). The end effector 110 includes a first rigid end 118 and a second rigid end 120 spaced apart longitudinally from the first rigid end 118. The first rigid end 118 is closer to the distal end 108 of the deployment arm 104 while the second rigid end 120 is closer to the proximal end 106 of the deployment arm 104. The TCP tendon 116 includes a first end 122 coupled to the first rigid end 118 and a second end 124 coupled to the second rigid end 120 such that each TCP tendon 116 extends between the first and second rigid ends 118, 120 of the end effector 110.

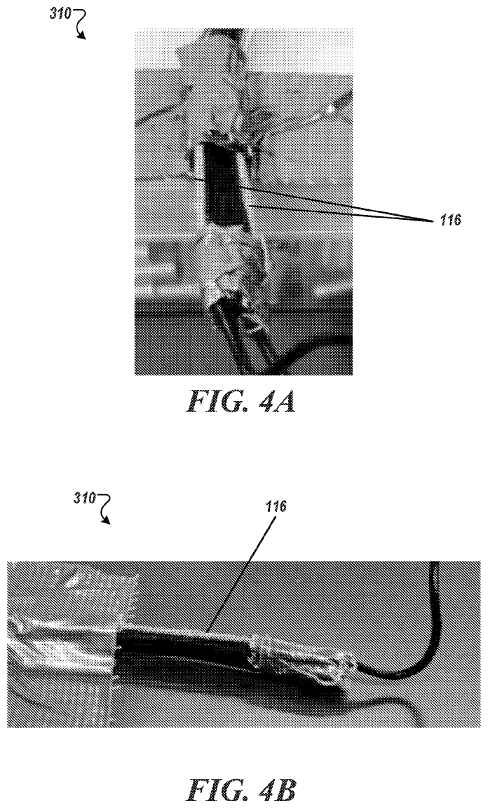

FIG. 3 shows another example end effector 210 having three TCP tendons 116 extending between a first rigid end 118 and a second rigid end 120. FIGS. 4A and 4B show different views of another example end effector 310 having two TCP tendons 116.

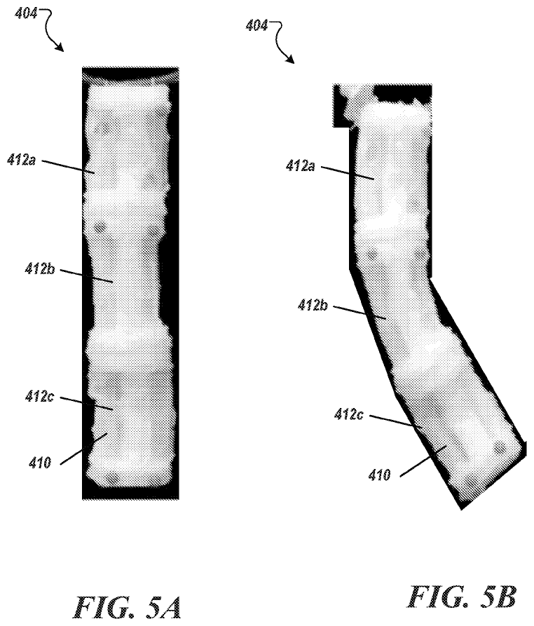

FIGS. 5A and 5B show another example of a deployment arm 404 (similar to deployment arm 104 of FIG. 2A). The deployment arm 404 includes three arm segments 412, labeled 412a, 412b, and 412c. The arm segment 412a is closer to the proximal end of the deployment arm 404 and an associated base system (not shown), while the arm segment 412c is further from the base system at the distal end of the deployment arm 404. The arm segment 412c is also the end effector 410 of the deployment arm 404 since it is coupled to the distal end of the deployment arm 404. Each of the three arm segments 412 in deployment arm 404 include TCP tendons (not shown), similar to that of end effectors 110, 210, and 310. Each of the TCP tendons of the deployment arm 404 is internal and thus not shown in FIGS. 5A and 5B.

In use, each of the TCP tendons in each arm segment 412 are configured to controllably expand and contract upon application of heat. Each TCP tendon is in electrical communication with the base system (e.g., base system 150 of FIG. 2A). In some examples, each arm segment 412 is in electrical communication with the base system individually (e.g., via individual wires extending along the deployment arm 404 from the corresponding arm segment 412 to the base system), while in other examples each arm segment 412 is in electrical communication with the base system and each other arm segment 412 (e.g., via a wire extending along the deployment arm 404 from the distal end to the proximal end and to the base system).

FIG. 5A shows the deployment arm 404 and associated TCP tendons in a neutral configuration. However, upon activation or application of a current, the TCP tendons extend to a length greater than the neural configuration. Therefore, in FIG. 5B, at least one TCP tendon in each of the arm segments 412b and 412c has expanded on one side. Thus, the deployment arm 404 in FIG. 5B is in the curved configuration.

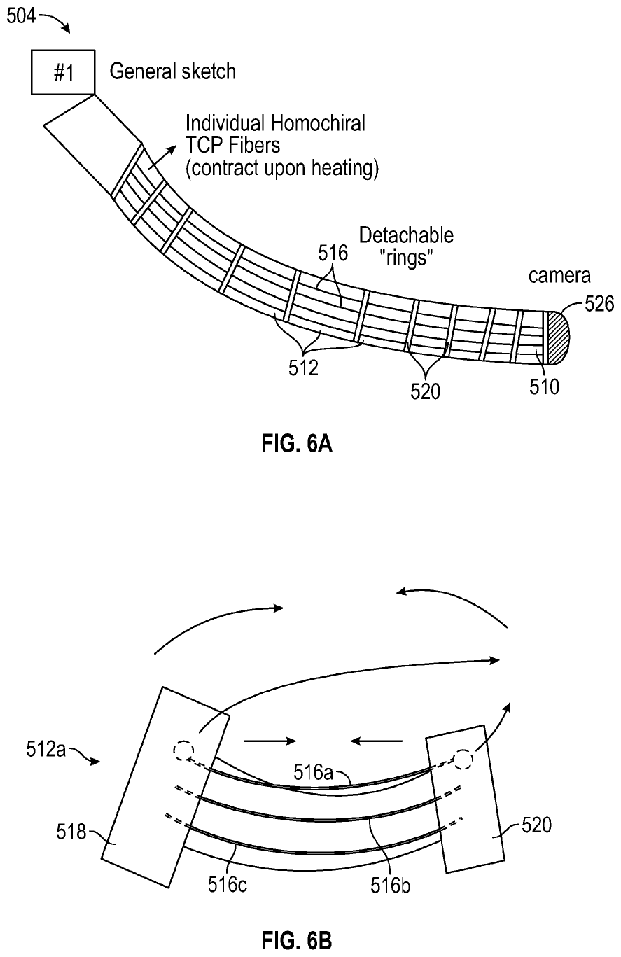

FIGS. 6A and 6B show another example deployment arm 504 having multiple arm segments 512 with an end effector 510 as the distal arm segment. TCP tendons 516 extend along the length of the deployment arm 504 through each of the arm segments 512. The arm segments 512 are separated by rigid ends 520. Additionally, the deployment arm 504 includes a camera 526 coupled to the end effector 510.

FIG. 6B shows a single arm segment 512a having three TCP tendons 516a, 516b, and 516c disposed between a first rigid end 518 and a second rigid end 520. As shown the arm segment 512a is in the curved configuration such that the TCP tendon 516a is contracted and/or the TCP tendon 516c is expanded.

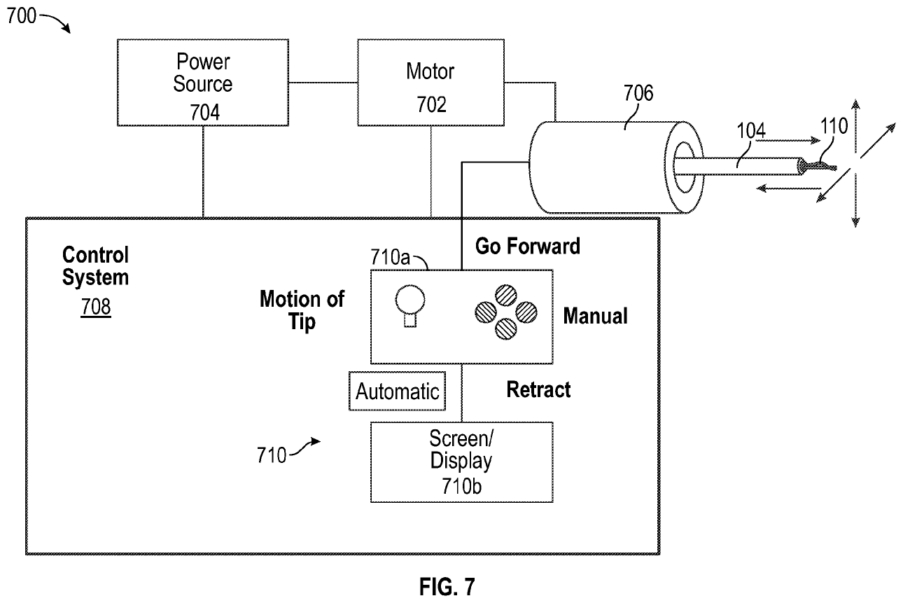

While the TCP tendons provide for movement of the end effector radially (e.g., an x-direction and y-direction), the deployment arm still requires longitudinal actuation (e.g., feeding or deploying the deployment arm in a z-direction down the patient's airway). The base system of the device provides such longitudinal actuation in addition to other control systems. Therefore, the combination of radial (or x-y) movement from the TCP tendons, along with longitudinal (or z) movement from the base system also for movement of the deployment arm or end effector in at least 3 degrees of freedom as it navigates the patient's airway (e.g., see the arrows of FIG. 7).

The base system 150 of device 100 (e.g., as shown in FIG. 2A) is shown in another implementation in FIG. 7 as base system 700. The base system 700 includes a motor 702 in electrical communication with a power source 704. The base system 700 further includes a driver 706 coupled to the motor 702 and the deployment arm 104. The power source 704 and motor 702 may be external to the driver 706 or internal to the driver 706. The driver 706 is configured to extend and retract the deployment arm 104 with respect to the base system 700 (as shown by the directional arrows adjacent to the deployment arm 104).

The base system 700 further includes a control system 708 including a user interface 710 (e.g., a physical user interface 710a with button or a screen/display 710b). The control system 708 is configured to receive input from a user (e.g., a medical professional) on the user interface 710 and to control the deployment arm 104 and the driver 706 in response to signals sent from the control system 708 to the device. For example, the user interface 710 includes controls for extending and retracting the deployment arm 104 via actuation of the motor 702 and/or driver 706. Additionally, the user interface 710 includes controls for moving the end effector 110 radially with respect to the deployment arm 104 (e.g., in the directions indicated by the arrows adjacent to the end effector). In some implementations, the driver 706 extends or retracts the deployment arm 104 while, simultaneously, the end effector 110 moves radially with respect to the deployment arm 104. In some implementations, the driver, motor, control system, and power source are all contained within a single housing to form the base system.

In implementations having a camera (e.g., end effector 510 with camera 526 of FIG. 6A), the screen/display 710b is in electrical or wireless communication with the camera. Thus, the control system 708 is configured to display on the screen/display 710b the location of the deployment arm 104 and/or end effector 110 within a patient's airway.

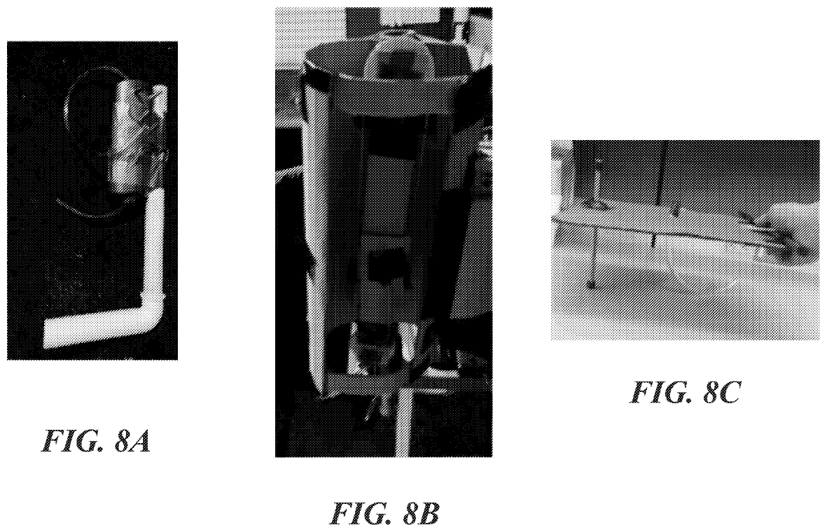

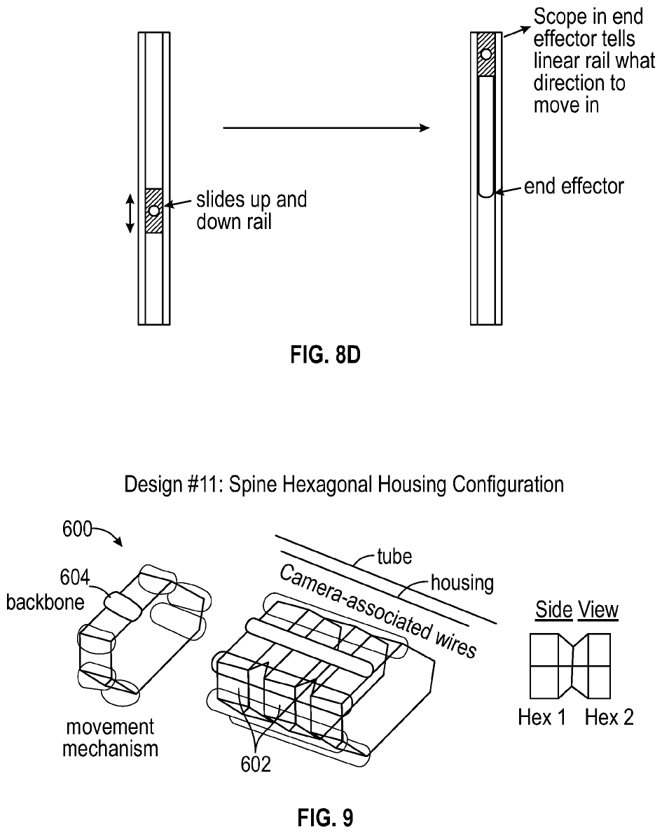

FIGS. 8A-8C show a variety of prototype devices for clamping the base system and driving the deployment arm. A wheel driven prototype is shown in FIG. 8A. For example, the driver 706 of FIG. 7 includes a wheel-driven system (not shown) wherein the deployment arm 104 is disposed between two or more wheels. One or more of the two or more wheels are powered and controlled by the motor 702 and the control system 708 to turn to dispense or retract the deployment arm 104.

In other implementations, the driver includes a belt-driven system similar to the wheel-driven system to extend or retract the deployment arm. An example belt driven prototype is shown in FIG. 8B. In other implementations, a lead screw system is used to extend or retract the deployment arm. An example lead screw prototype is shown in FIG. 8C.

FIG. 8D shows an example of an alternative deployment system for the base system. Rather than use a wheel- or belt-driven system, FIG. 8D provides a rail deployment system. In this example, the deployment arm and end effector slide back and forth along the rail during an intubation operation while the end effector orients the deployment arm within the patient's airway.

FIG. 9 shows a perspective and a side view of a hexagonal spine housing 600. The housing includes several segments 602 connected to each other along a backbone 604. The deployment arm, the flexible tube, and wires are disposed within the housing 600 extending from the base system to the end effector. The housing 600 is configured to expand and contract depending on the extension or contraction of the deployment arm disposed therein. The housing 600 is made of biocompatible material which separates the inner components from contacting the patient until such time as the flexible intubation tube is deployed. The housing 600 is thermodynamically favorable to prevent overheating of components, and the housing is reusable and sterilizable.

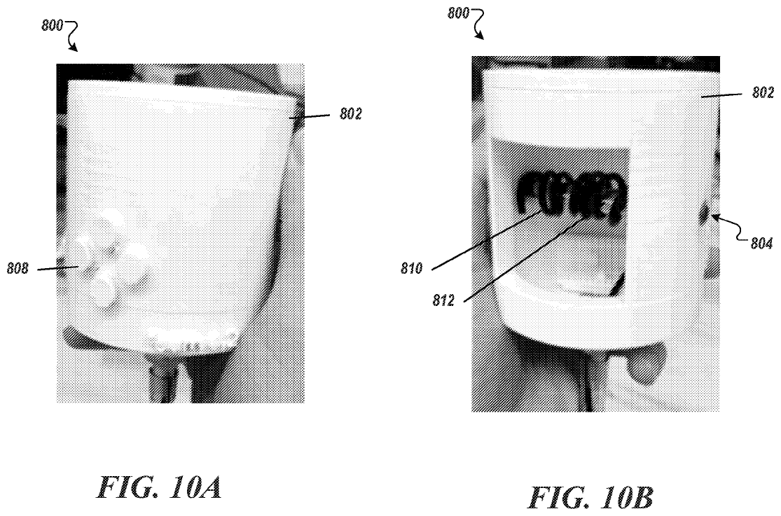

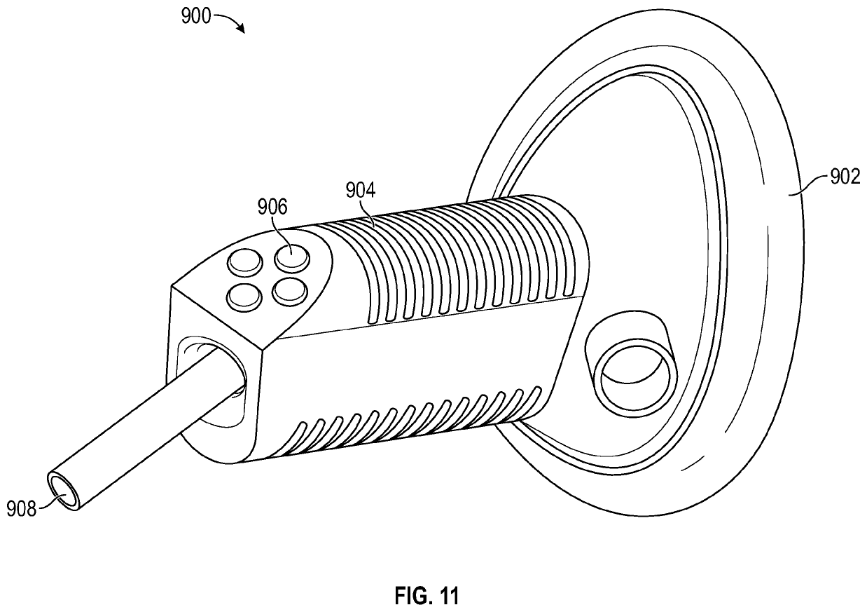

FIGS. 10A and 10B show another prototypical implementation of the base system, shown as base system 800. Base system 800 includes an outer housing 802 with buttons 808 for controlling one or more of the deployment arm 804 and driver. The outer housing 802 includes a cutout for visibility of the interior in testing. The base system 800 includes a twisted and coiled polymer (TCP) actuation system 810 which is coiled around a center shaft 812. Upon actuation, the TCP actuation system 810 extends out of the housing 802 and into a patient's airway.

FIG. 11 shows an implementation of the intubation device 100, shown as intubation device 900 having a face mask portion 902 engageable over a patient's mouth and/or nose. In FIG. 11, the base system 904 is integrally coupled to the face mask portion 902 The base system 904 includes control buttons 906 disposed on an outer surface of the base system 904 and configured to control operation of the deployment arm 908. The deployment arm 908 is pulled into the base system 904 before extending through the face mask portion 902 and into the patient's airway.

In various implementations, the base system is clamped or secured to a rigid object such that the intubation device will not move around excessively during an intubation procedure. In some implementations, the base system includes a stabilizer including at least one of a handheld brace, a c-clamp brace, and a stabilization board dimensioned to accommodate a patient's head. FIG. 8A shows a prototype device including a handheld stabilizer. In this example, a user would hold the stabilizer (e.g., a handle) attached to the base system to ensure consistent location of the device during intubation. The handheld stabilizer represents a quick, intuitive, and cost-effective stabilizing solution. FIG. 8B shows a prototype device including a c-clamp brace. The c-clamp may engage with a bed rail or any other rigid object adjacent to the patient during intubation. FIG. 8C shows a tripod stabilizer configured to hold the device over the patient during intubation.

The examples described herein have recited control systems, buttons, and cameras facilitating manual intubation. However, the systems, methods, and devices described herein have automatic capabilities as well. Each of the above-described examples may be implemented in an automated system wherein the individual motions of the end effector, deployment arm, and or the base system are controlled with limited or no input from a human user. In some implementations, a user (e.g., medical professional or device operator) may have control over a selection of components. For example, the user may guide the longitudinal extension of the deployment arm into the patient's airway while an automated system controls the end effector's path through the airway (e.g., the curvature required to intubate).

In other implementations, the device automatically deploys and navigates with a user ready to stop the device when needed. In other implementations, the control system uses artificial intelligence and machine learning to complete the intubation process. For example, a control system may be trained on the anatomy of an airway and how to deploy a flexible intubation tube. The device may use a known set of anatomical landmarks and/or previous intubation results to navigate a patient's airway. Such a system can be more consistent and efficient compared to manual intubation.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. An automated endotracheal intubation device, comprising:
a flexible tube sized to be advanced within a patient's airway;
a base system; and
a deployment arm having a proximal end coupled to the base system and a distal end spaced apart from the proximal end, the deployment arm comprising:
at least one arm segment coupled to and extending from the base system, the at least one arm segment defining a channel within which the flexible tube is disposed; and
an end effector coupled to the distal end of the deployment arm, the end effector comprising one or more twisted and coiled polymer (TCP) tendons configured to controllably expand and contract upon application of heat.

2. The device of claim 1, wherein the base system comprises:
a stabilizer comprising at least one of a handheld brace, a c-clamp brace, and a stabilization board dimensioned to accommodate a patient's head.

3. The device of claim 1, wherein the base system comprises:
a motor in electrical communication with a power source; and
a driver coupled to the motor and the deployment arm, the driver configured to extend and retract the deployment arm with respect to the base system.

4. The device of claim 3, wherein the base system further comprises a control system comprising a user interface, the control system configured to receive input from a user on the user interface and control the deployment arm and the driver in response to signals sent from the control system to the device.

5. The device of claim 3, wherein the driver comprises a belt-driven system or a wheel-driven system.

6. The device of claim 4, further comprising a camera coupled to the end effector; and
a screen in electrical or wireless communication with the camera,
wherein the control system is configured to display on the screen a location of the deployment arm and/or end effector within a patient's airway.

7. The device of claim 1, wherein the at least one arm segment comprises a plurality of arm segments, each of the plurality of arm segments comprising one or more twisted and coiled polymer (TCP) tendons.

8. The device of claim 1, wherein the deployment arm includes one or more twisted and coiled polymer (TCP) tendons extending along the at least one arm segment or the end effector, the one or more TCP tendons configured to controllably move the at least one arm segment or the end effector between a neutral configuration and a curved configuration with 1 degree of freedom.

9. The device of claim 8, wherein the deployment arm includes a plurality of twisted and coiled polymer (TCP) tendons extending along the at least one arm segment or the end effector, the plurality of TCP tendons configured to controllably move the at least one arm segment or the end effector between the neutral configuration and a plurality of curved configurations with at least 2 degrees of freedom.

10. The device of claim 9, wherein the base system comprises:
a motor in electrical communication with a power source; and
a driver coupled to the motor and the deployment arm, the driver configured to extend and retract the deployment arm with respect to the base system,
wherein the driver configured to extend and retract the deployment arm with respect to the base system operates simultaneously with the plurality of TCP tendons to move the deployment arm and the end effector in at least 3 degrees of freedom to navigate the patient's airway.

11. A method of automated endotracheal intubation, the method comprising:
providing an intubation device including a base system and a deployment arm coupled to the base system, the base system comprising a driver system for extension, and the deployment arm comprising a twisted and coiled polymer (TCP) end effector;
inserting the deployment arm into a patient's mouth adjacent to an airway;
activating the driver system to extend the deployment arm further into the airway;
activating the twisted and coiled polymer (TCP) end effector to navigate a patient's anatomy;
deploying a flexible tube configured to maintain the patient's airway;
retracting the end effector and the deployment arm out of the patient's airway.

12. The method of claim 11, further comprising placing a stabilizer underneath a patient's head.
